(19) United States
(12) Patent Application Publication
(10) Pub. No.: US 2013/0042868 A1
(43) Pub. Date: Feb. 21, 2013

(54) HIGH FREQUENCY OSCILLATION VENTILATOR CONTROL SYSTEM

(71) Applicant: Yong Liu, Yorba Linda, CA (US)

(72) Inventor: Yong Liu, Yorba Linda, CA (US)

(21) Appl. No.: 13/212,157

(22) Filed: Aug. 17, 2011

(51) Int. Cl. A61M 16/00

(52) U.S. Cl. 128/204.21

(57) ABSTRACT

A high frequency oscillation ventilator including an oscillating piston control system and a mean airway pressure control system. The oscillating piston control system and the mean airway pressure control system are closed-loop control systems. The oscillating piston control system is independent of the mean airway pressure control system.

### BACKGROUND

Typically, high frequency oscillation (HFO) ventilators have a plurality of open-loop control systems that are dependent on one another. For example, if it is desired to increase the oscillation pressure amplitude on a HFO ventilator, then a medical practitioner is required to manually adjust a pressure amplitude controller via a dial. Accordingly, other parameters of the HFO ventilator that are dependent on the pressure amplitude automatically change due to the adjustment of the pressure amplitude by the medical practitioner. Therefore, the medical practitioner has to adjust other parameters simultaneously.

### BRIEF DESCRIPTION OF THE DRAWINGS

FIG. 1 illustrates an example of a HFO ventilator, in accordance with an embodiment of the present invention.

FIG. 2 illustrates an example of a MAP control system, in accordance with an embodiment of the present invention.

FIG. 3 illustrates an example of a bias flow control system, in accordance with an embodiment of the present invention.

FIG. 4 illustrates an example of a method for controlling a HFO ventilator, in accordance with an embodiment of the present invention.

The drawings referred to in this description should be understood as not being drawn to scale except if specifically noted.

### DESCRIPTION OF EMBODIMENTS

Reference will now be made in detail to embodiments of the present technology, examples of which are illustrated in the accompanying drawings. While the technology will be described in conjunction with various embodiment(s), it will be understood that they are not intended to limit the present technology to these embodiments. On the contrary, the present technology is intended to cover alternatives, modifications and equivalents, which may be included within the spirit and scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

In general, HFO ventilators employ an active ventilation in which gas is pushed into and pulled out of a patient's lungs during alternate cycles of the oscillating piston of the ventilator. One motion of the piston creates a positive-going pressure relative to the static pressure in the patient's airway. As the motion of the piston moves in an opposite direction, the dynamic pressure generated reverses from positive-going to negative-going. Accordingly, the generated bi-polar dynamic pressure waveform provides respiratory gas exchange.

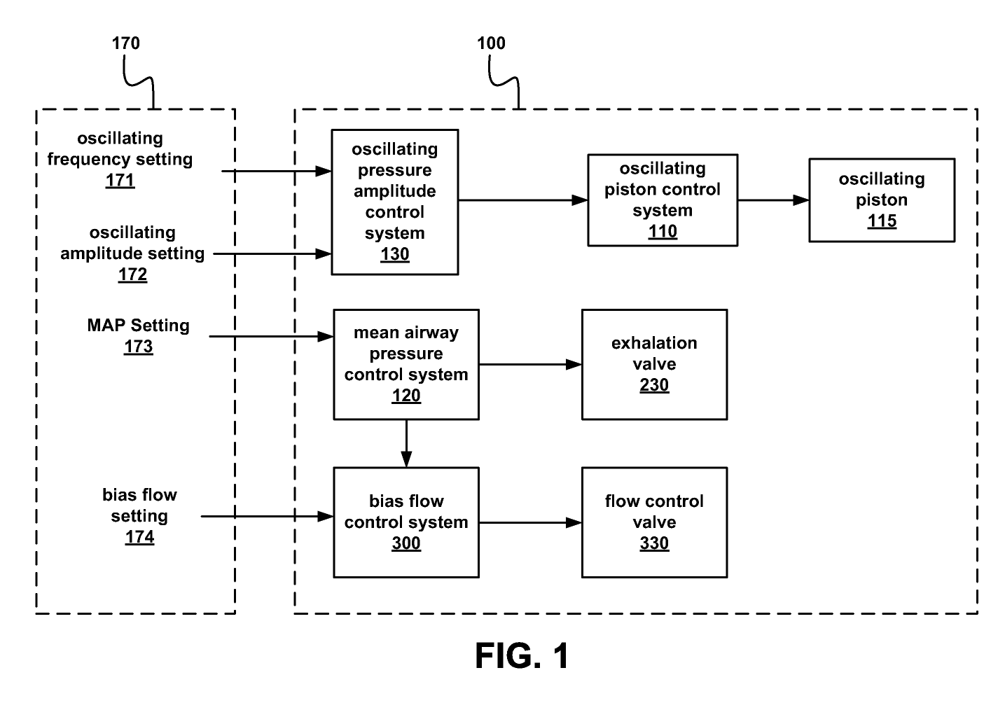

FIG. 1 depicts an embodiment of HFO ventilator 100. A discussion regarding embodiments of HFO ventilator 100 is provided below. First, the discussion will describe the structure or components of various embodiments of HFO ventilator 100. Then the discussion will describe the operational description of HFO ventilator 100.

HFO ventilator 100 includes oscillating piston control system 110, mean airway pressure (MAP) control system 120, oscillating pressure amplitude control system 130 and bias flow control system 300.

Oscillating piston control system 110 is configured to control oscillating piston 115. A neutral position of oscillating piston 115 is maintained. In one embodiment, oscillating piston 115 generates an oscillating pressure between 3 Hertz (Hz) and 20 Hz.

Oscillating piston control system 110 controls oscillating piston 115 to generate an oscillating waveform with high order harmonic frequencies other than base line setting frequency. The generated oscillating waveform can be, but is not limited to a square waveform and sinusoidal waveform. It should be appreciated that HFO ventilator 100 can tune the shape of the waveform.

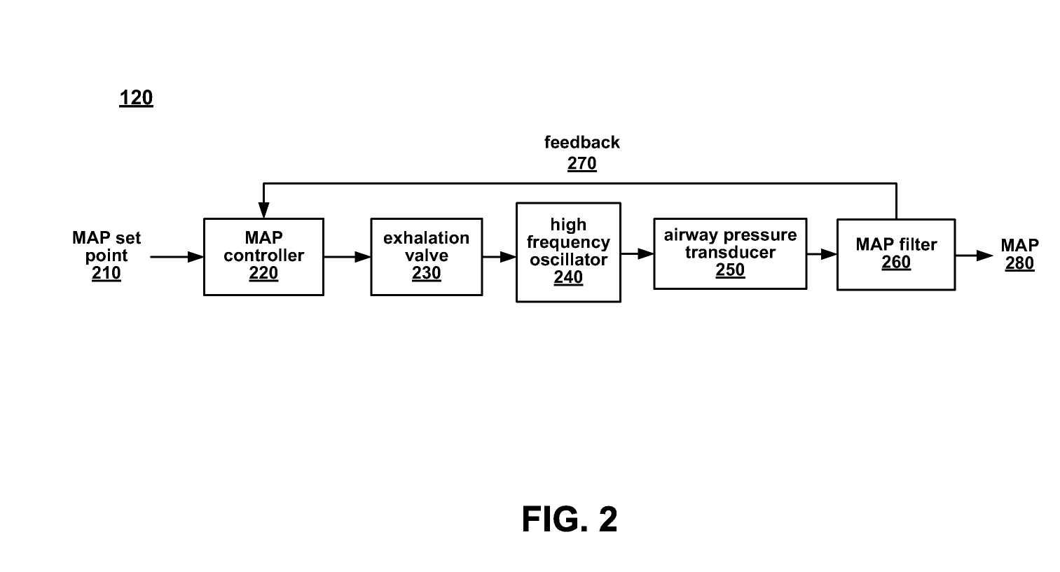

MAP control system 120 is configured to control mean airway pressure of HFO ventilator 100. Mean airway pressure is the average pressure over one inspiration/expiration cycle. In particular, MAP control system 120 controls exhalation valve 230. An embodiment of MAP control system 120 is depicted in FIG. 2, which is described in detail below.

Oscillating pressure amplitude control system 130 is configured to control the oscillating pressure amplitude of HFO ventilator 100. In one embodiment, an oscillating pressure amplitude is at least 5 cmH20. In another embodiment, an oscillating pressure amplitude with accuracy less than 1 cmH20.

In various embodiments, oscillating piston control system 110, MAP control system 120, oscillating pressure amplitude control system 130 and bias flow control system are closed-loop systems. In other words, oscillating piston control system 110 includes a feedback loop that facilitates in controlling oscillating piston 115, MAP control system 120 includes a feedback loop that facilitates in controlling mean airway pressure, and oscillating pressure amplitude control system 130 includes a feedback loop that facilitates in controlling the oscillating pressure amplitude.

In contrast, in conventional ventilators, control systems for various parameters (e.g., pistons, mean airway pressure, pressure amplitudes) are open loop systems.

In various embodiments, oscillating piston control system 110, MAP control system 120, oscillating pressure amplitude control system 130 and bias flow control system 300 are independent (e.g., decoupled) from one another. In other words, each of the control systems can be adjusted independently from one another. For example, if the frequency of the oscillating piston was adjusted, then it is guaranteed that the same amplitude of oscillation pressure is delivered to the patient. In another example, HFO 100 delivers oscillation pressure amplitude to a patient independent of a MAP setting.

In particular, settings 170 can be adjusted independently from one another. For example, oscillating frequency setting 171, oscillating amplitude setting 172, MAP setting 173 and bias flow setting 174 can be adjusted independently from one another.

FIG. 2 depicts an embodiment of MAP control system 120. MAP control system 120 includes MAP controller 220, exhalation valve 230, high frequency oscillator 240, airway pressure transducer 250, and MAP filter 260.

During use of HFO ventilator 100, a MAP set point 210 is provided to MAP control system 120. Accordingly, MAP 280 is adjusted based, in part, on feedback 270.

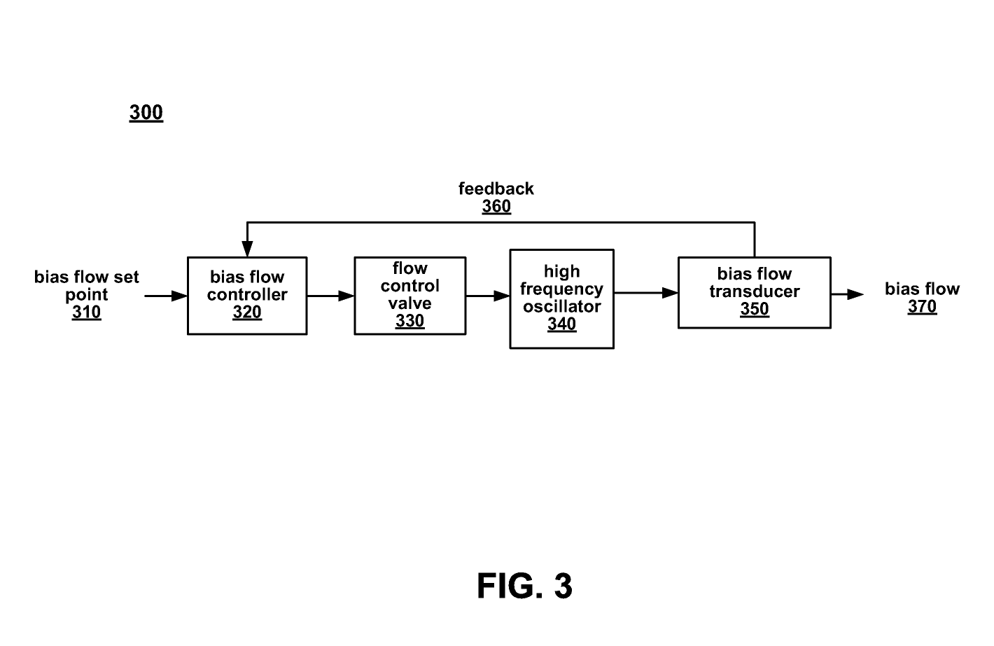

FIG. 3 depicts an embodiment of bias flow control system 300. Bias flow control system 300 includes bias flow controller 320, flow control valve 330, high frequency oscillator 340, and bias flow transducer 350. In particular, bias flow control system 300 controls flow control valve 330.

During use of HFO ventilator 100, bias flow set point 310 is provided to bias flow control system 300. Accordingly, bias flow 370 is adjusted based, in part, on feedback 360. In general, bias flow 370 is the rate at which the flow of gas, through the oscillator, is delivered to the patient.

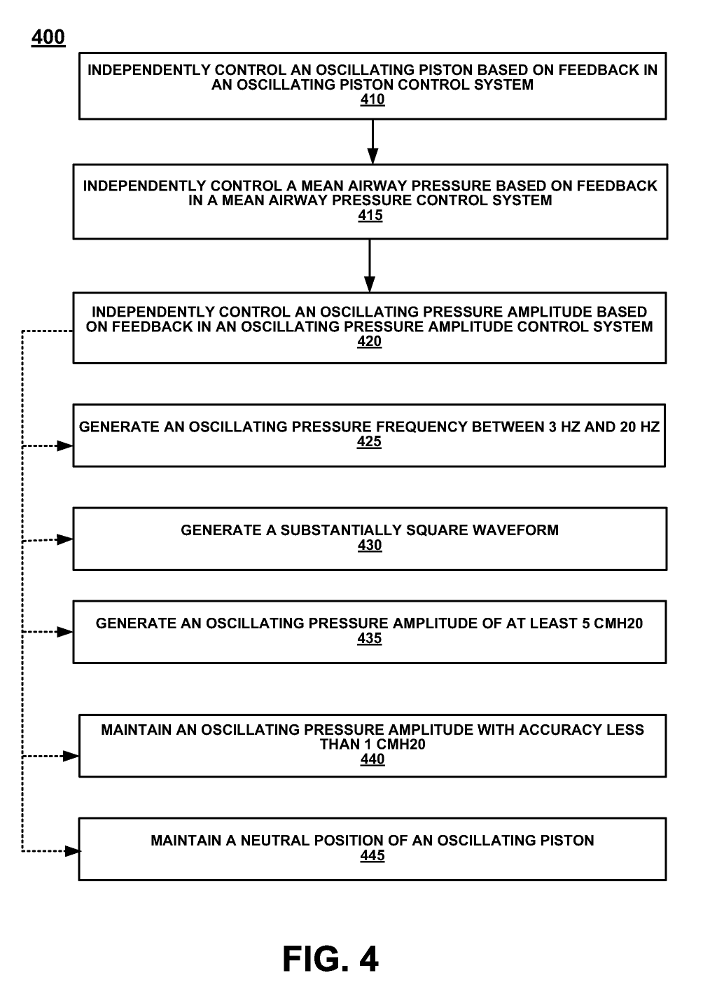

FIG. 4 depicts method 400 for controlling a high frequency oscillation ventilator, in accordance with an embodiment of the present invention. In various embodiments, method 400 is carried out by processors and electrical components under the control of computer readable and computer executable instructions. The computer readable and computer executable instructions reside, for example, in a data storage medium such as computer usable volatile and non-volatile memory. However, the computer readable and computer executable instructions may reside in any type of computer readable storage medium. In some embodiments, method 400 is performed at least by HFO ventilator 100, as described in FIG. 1.

At 410, an oscillating piston is independently controlled based on feedback in an oscillating piston control system. For example, oscillating piston 115 is independently controlled by close-loop oscillating piston control system 110.

At 415, a mean airway pressure is independently controlled based on feedback in a mean airway pressure control system. For example, mean airway pressure 280 is independently controlled based on feedback 270 in a MAP control system 120.

At 420, independently control an oscillating pressure amplitude based on feedback in an oscillating pressure amplitude control system. For example, an oscillating pressure amplitude is based on a feedback generated in close-loop oscillating pressure amplitude control system 130.

At 425, an oscillating pressure frequency is generated between 3 Hz and 20 Hz. At 430, a substantially square waveform is generated. It should be understood that a waveform is generated such, but not limited to, a sinusoidal waveform. At 435, an oscillating pressure amplitude of at least 5 cmH20 is generated. At 440, an oscillating pressure amplitude accuracy is maintained less than 1 cmH20. At 445, a neutral position of an oscillating piston is maintained.

Various embodiments of the present invention are thus described. While the present invention has been described in particular embodiments, it should be appreciated that the present invention should not be construed as limited by such embodiments, but rather construed according to the following claims.

1. A high frequency oscillation ventilator comprising:
an oscillating piston control system; and
a mean airway pressure control system, wherein said oscillating piston control system and said mean airway pressure control system are closed-loop control systems, and wherein said oscillating piston control system is independent of said mean airway pressure control system.

2. The high frequency oscillation ventilator of claim 1, further comprising:
an oscillating pressure amplitude control system, wherein said oscillating pressure amplitude control system is a closed loop control system, and wherein said oscillating pressure amplitude control system is independent of said oscillating piston control system and said mean airway pressure control system.

3. The high frequency oscillation ventilator of claim 1, further comprising:
an oscillating pressure frequency between 3 Hz and 20 Hz.

4. The high frequency oscillation ventilator of claim 1, further comprising:
an oscillating pressure amplitude is at least 5 cmH20.

5. The high frequency oscillation ventilator of claim 1, wherein said oscillating piston control system comprises:
a self-centering oscillating piston.

6. The high frequency oscillation ventilator of claim 1, further comprising:
a flow control valve.

7. The high frequency oscillation ventilator of claim 1, further comprising:
an exhalation valve.

8. A method for controlling a high frequency oscillation ventilator, said method comprising:
independently controlling an oscillating piston based on feedback in an oscillating piston control system; and
independently controlling a mean airway pressure based on feedback in a mean airway pressure control system.

9. The method of claim 8, further comprising:
independently controlling an oscillating pressure amplitude based on feedback in an oscillating pressure amplitude control system.

10. The method of claim 8, further comprising:
generating an oscillating pressure frequency between 3 Hz and 20 Hz.

11. The method of claim 8, further comprising:
generating a substantially square waveform.

12. The method of claim 8, further comprising:
generating an oscillating pressure amplitude of at least 5 cmH20.

13. The method of claim 8, further comprising:
maintaining an oscillating pressure amplitude accuracy less than 1 cmH20.

14. The method of claim 8, further comprising:
maintaining a neutral position of an oscillating piston.
